(19) United States
(12) Patent Application Publication
(10) Pub. No.: US 2004/0019243 A1
(43) Pub. Date: Jan. 29, 2004

(54) 1,3-Bis(trifluoromethyl)benzene derivatives

(76) Inventors: Peter David Nightingale, Stockport (GB); Reginald Barry O'Neill, Oldham (GB); Robert Rhodes, Chesterfield (GB)

Correspondence Address: NOTARO AND MICHALOS, 100 DUTCH HILL ROAD, SUITE 110, ORANGEBURG, NY, 10962-2100, US

(21) Appl. No.: 10/381,238

(22) PCT Filed: Sep. 24, 2001
(86) PCT No.: PCT/GB01/04258

(30) Foreign Application Priority Data
Sep. 23, 2000 (GB) 0023383.3

(51) Int. Cl. C07C 25/13

(52) U.S. Cl. 570/144

(57) ABSTRACT

A method of manufacture of 3,5-bis(trifluoromethyl)bromobenzene, comprising the addition of a brominating reagent to a mixture of 3,5-bis(trifluoromethyl)benzene together with at least one of sulphuric acid or oleum in the absence of acetic acid. A method of production of 3,5-bis(trifluoromethyl)acetophenone comprising the reaction of 3,5-bis(trifluoromethyl)phenyl magnesium bromide with acetyl chloride in the presence of cuprous chloride. A method of production of 3,5-bis(trifluoromethyl)acetophenone comprising the steps of reacting 3,5-bis(trifluoromethyl)phenyl magnesium bromide with acetic anhydride, adding water, and recovering the product by azeotropic distillation. A method of removal of impurities including 3,5-bis(trifluoromethyl)acetate, 4-bromobutyl acetate and 4-chlorobutyl acetate from a preparation of 3,5-bis(trifluoromethyl)acetophenone, the method comprising heating the 3,5-bis(trifluoromethyl)acetophenone with a dilute solution of alkali. A method of production of 3,5-bis(trifluoromethyl)phenyl magnesium bromide comprising the reaction of 3,5-bis(trifluoromethyl)bromobenzene with magnesium in a solvent whilst maintaining the temperature of the reactants above 20° C. and below the reflux temperature of the solvent.

The present invention relates to a process of producing 1,3-bis(trifluoromethyl)benzene derivatives substituted in the 5-position and, in particular, 3,5-bis(trifluoromethyl)bromobenzene and 3,5-bis(trifluoromethyl)acetophenone. These compounds are useful intermediates in pharmaceutical manufacture. 3,5-bis(trifluoromethyl)bromobenzene is a very versatile intermediate, but its use is restricted owing to problems in its manufacture. 1,3-bis(trifluoromethyl)benzene is a useful starting material in the manufacture of this compound and 3,5-bis(trifluoromethyl)acetophenone in that it is readily available on a large scale. Bromination of 1,3-bis(trifluoromethyl)benzene is difficult owing to the relative unreactivity of the benzene nucleus to conventional bromination. In order to overcome this problem processes are known using expensive solvents such as trifluoroacetic acid. This material causes environmental problems on disposal.

Bromination in sulphuric acid is also reported in which sulphuric acid is added to a mixture of 1,3-dibromo-5,5-dimethylhydantoin in 1,3-bis(trifluoromethyl)benzene. This process is not suitable for industrial use owing to problems in mixing and heat transfer. Furthermore, solvent extraction is required to isolate the product and the use of the large amounts of solvent required on an industrial scale would be environmentally undesirable. It is further noted that the waste products of the reaction are environmentally unfavourable.

The present invention seeks to provide an alternative method of producing 3,5-bis(trifluoromethyl)bromobenzene which addresses these problems.

According to the present invention there is provided a method of making 3,5-bis(trifluoromethyl)bromobenzene by adding a brominating reagent to a mixture of 1,3-bis(trifluoromethyl)benzene together with at least one of sulphuric acid or oleum in the absence of acetic acid.

The preferred brominating reagent includes any of the following: elemental bromine, N-bromosuccinimide (NBS) and 1,3-dibromo-5,5dimethylhydantoin (DBDMH).

In a preferred embodiment of the invention at least 3 parts by weight of acid/oleum, more preferably 4 parts by weight, is used to one part of the 1,3-bis(trifluoromethyl)benzene. Using a lower ratio of acid/oleum to 1,3-bis(trifluoromethyl)benzene typically results in a mixture that is difficult to stir, whereas at the preferred levels mentioned above, the reaction mixture is more easily agitated. This helps to prevent localised pockets of reaction that could result in bromination. In addition, the preferred ratio of at least 3 parts by weight of acid/oleum to 1,3-bis(trifluoromethyl)benzene is advantageous in that the product may be separated from the acid/oleum without the need for dilution with water, thus preventing the need for a highly exothermic dilution step and helping to reduce the volume of waste material generated.

Preferably, the acid is not diluted to less than 90% solution. The acid is ideally concentrated. Commercially available concentrated acid is normally understood to be 96 to 98% solution.

Controlled, portion-wise addition of the brominating reagent to 1,3-bis(trifluoromethyl)benzene in the presence of sulphuric acid or oleum substantially avoids the problems, due to poor agitation and heat transfer, associated with the prior art method. In particular, the portion-wise addition of brominating reagent allows the reaction temperature to be controlled, which is especially important in large-scale production of 3,5-bis(trifluoromethyl)bromobenzene. Furthermore, the product can be separated without the need for solvent extraction and instead by a simple separation from the solution of spent brominating agent in sulphuric acid. Furthermore, the waste solution is less environmentally unfavourable than trifluoroacetic acid. For example, when the bromination reagent is DBDMH then the waste solution is dimethylhydantoin (DMH) in sulphuric acid.

Preferably, the reaction is carried out using 1 equivalent of brominating reagent. Whilst this may lead to incomplete conversion of the starting material, it has the advantage of keeping down the levels of unwanted side products, such as unwanted isomers and multibrominated compounds. In addition, this method is economically advantageous in that expensive brominating reagent is not wasted.

The reaction is ideally carried out at a temperature in the range from −10° C. to 30° C. and preferably from 0° C. to 10° C. and still more preferably from 3° C. to 5° C. Reaction at such low temperatures reduces the formation of unwanted isomers and multibrominated compounds and provides enhanced yields.

Colouration of the product caused by dissolved bromine may be removed by a bisulphite wash. After traces of bromine have been removed by suitable washing techniques the product is generally purified by fractional distillation.

By this method 3,5-bis(trifluoromethyl)bromobenzene can be produced in high yield and purity; in fact the purity may be in excess of 99%. Any unreacted 1,3-bis(trifluoromethyl)benzene may be recycled to the next reaction batch.

3,5-bis(trifluoromethyl)bromobenzene is especially useful in the manufacture of 3,5-bis(trifluoromethyl)acetophenone using Grignard chemistry, but the use of this technique has been limited owing to poor yields in the conventional process and low purity products.

Grignard reagents are known to be versatile intermediates in the preparation of a wide variety of downstream products. The use of Grignard reagents to produce carbonyl compounds is, however, known to be difficult owing to further reaction leading to the formation of alcohols. For this reason it is common to convert the Grignard reagent to another organometallic reagent in situ, normally a cadmium derivative. Such materials are expensive and lead to environmental problems.

It has been known to produce 3,5-bis(trifluoromethyl)acetophenone using a multistage reaction from 3,5-bis(trifluoromethyl)phenyl magnesium bromide. Initially, this is reacted with solid carbon dioxide to yield 3,5-bis(trifluoromethyl)benzoic acid which in turn can be converted to 3,5-bis(trifluoromethyl)benzoyl chloride. The reaction of this material with organocopper reagents at −78° C. provides 3,5-bis(trifluoromethyl)acetophenone but leads also to the production of large quantities of environmentally unfavourable copper and lithium salts as waste. The 3,5-bis(trifluoromethyl)acetophenone is removed via solvent extraction. The process as a whole is inefficient, expensive and environmentally unfriendly.

A further object of the present invention is to provide a process for the production of 1,3-bis(trifluoromethyl)benzene derivatives that is efficient and commercially useful.

According to a second aspect of the present invention therefore there is provided a method of producing 3,5-bis(trifluoromethyl)acetophenone by reacting 3,5-bis(trifluoromethyl)phenylmagnesium bromide with acetyl chloride in the presence of cuprous chloride.

The cuprous chloride may be present in catalytic amounts or in equimolar amounts to the acetyl chloride. In the presence of catalytic amounts of cuprous chloride, the reaction temperature is preferable in the range from 30° C. to 40° C. In the presence of equimolar amounts of cuprous chloride, however, the reaction temperature is preferably maintained below 30° C.

Preferably, the Grignard reagent is reacted with acetyl chloride in an organic solvent, most preferably tetrahydrofuran (THF).

The ketone derivative can be isolated from the reaction mixture in high yield and purity. Conventional techniques such as solvent extraction can be used.

The product is isolated by simple off distillation of the solvent from the water-quenched reaction mixture followed by steam distillation of the residue. The product is thus separated from high boiling inorganic and polymeric impurities. The product can be further purified by fractional distillation under reduced pressure, to achieve purity of approximately 99%.

According to a further aspect of the present invention there is provided a method of producing 3,5-bis(trifluoromethyl)acetophenone comprising the steps of reacting 3,5-bis(trifluoromethyl)phenylmagnesium bromide with acetic anhydride adding water, and recovering the product by azeotropic distillation.

The process of the invention is simpler, cheaper and provides a purer product than those of the prior art.

The addition of water serves to decompose any excess of acetic anhydride present in the mixture. Azeotropic distillation separates the 3,5-bis(trifluoromethyl)acetophenone from aqueous and high boiling point inorganic and organic impurities, including dimers.

Ideally, 3,5-bis(trifluoromethyl)phenylmagnesium bromide is reacted with acetic anhydride in an organic solvent, preferably THF, this being distilled off prior to azeotropic distillation to recover the product. Advantageously, the process of the present invention does not require extraction of the product into any further organic solvent, thus minimising the level of organic waste generated.

Preferably, a slight excess of acetic anhydride is used. Typically, less than around 1.5 equivalents of acetic anhydride are used, and most preferably less than 1.1 equivalents of acetic anhydride are used.

The reaction is preferably carried out at temperatures in the range from −15° C. to 15° C., most preferably from −10° C. to −5° C.

The product can be further purified by fractional distillation under reduced pressure. A yield of 70-80% based on the initial 3,5-bis(trifluoromethyl)bromobenzene may be expected.

Although highly pure 3,5-bis(trifluoromethyl)acetophenone can be produced using the method of the invention described above, some small amounts of impurities may be present in the final product. These impurities may arise from side reactions and/or partial degradation of the reaction solvent. The principal impurities are 3,5-bis(trifluoromethyl)phenyl acetate, which has the same boiling point as the main product, 4-bromobutyl acetate and 4-chlorobutyl acetate. These substances, which may be present at levels of up to 0.5%, are known to interfere with at least one downstream synthetic process in which the 3,5-bis(trifluoromethyl)acetophenone may be used.

A further object of the present invention therefore is to provide a method for the purification of 3,5-bis(trifluoromethyl)acetophenone that can substantially remove the aforementioned impurities.

According to a further aspect of the present invention therefore there is provided a method of removal of impurities including 3,5-bis(trifluoromethyl)phenyl acetate, 4-bromobutyl acetate and 4-chlorobutyl acetate from a preparation of 3,5-bis(trifluoromethyl)acetophenone, the method comprising heating the 3,5-bis(trifluoromethyl)acetophenone with a dilute solution of alkali.

Using this method, it is possible to reduce the level of the aforementioned impurities to below 0.05%.

Preferably, the 3,5-bis(trifluoromethyl)acetophenone is heated at reflux with the dilute solution of alkali.

The dilute solution of alkali may comprise any suitable alkali. Preferably, however, sodium hydroxide is used. Most preferably, an approximately 1N solution of sodium hydroxide is used. The quantity of alkali used may be varied depending upon the levels of impurity present.

The 3,5-bis(trifluoromethyl)acetophenone is preferably heated with the dilute solution of alkali for at least 30 minutes. The heating time may be extended without any deleterious effects on the products.

The production of the Grignard reagent, 3,5-bis(trifluoromethyl)phenylmagnesium bromide, from 3,5-bis(trifluoromethyl)bromobenzene can be carried out using conventional techniques. Reaction with finely divided magnesium is carried out under a nitrogen atmosphere under anhydrous conditions using the well known solvents including diethyl ether, dimethoxyethane and THF; especially preferred is THF. Conventionally, reactions are conveniently carried out at the reflux temperature of the solvent.

According to a further aspect of the present invention there is provided a method of producing 3,5-bis(trifluoromethyl)phenylmagnesium bromide by the reaction of 3,5-bis(trifluoromethyl)bromobenzene with magnesium in a solvent whilst maintaining the temperature of the reactants above 20° C. and below the reflux temperature of the solvent.

By maintaining the temperature below the reflux temperature of the solvent improved yields are obtained. Also, it has been found that if the reaction is carried out at reflux temperature, the magnesium becomes coated with a brown substance and the reaction stops, leading to incomplete utilisation of the 3,5-bis(trifluoromethyl)bromobenzene. Preferred solvents include any of diethyl ether, dimethoxyethane, butyldiglyme, 2-methyl THF and THF. Preferably, the temperature is maintained at between 30° C. and 60 C, more preferably between 35° C. and 50° C., and ideally at approximately 45° C. It has been found that at temperatures below 20° C. it is extremely difficult to achieve initiation of the reaction.

In order that the present invention may be more readily understood specific embodiments thereof are disclosed herein below by way of example only.

### EXAMPLE 1

1,3-Bis(trifluoromethyl)benzene (1 kg) was added to concentrated sulphuric acid (4 kg). The mixture was agitated and cooled to 5° C. DBDMH (668 g) was added over 4 hours keeping the temperature between 0° C. and 10° C. The mixture was allowed to separate and the organic phase washed with water and a dilute solution of sodium bisulphite. The product was fractionally distilled to give 3,5-bis(trifluoromethyl)bromobenzene 1100 g (80%) of 99% purity.

### EXAMPLE 2

3,5-Bis(trifluoromethyl)bromobenzene, (1630 g), in THF, (3 kg), was fed to a slurry of magnesium turnings (140 g) in THF (1 kg). The temperature was maintained at approximately 45° C. The solution of Grignard reagent was fed to a mixture of acetic anhydride (580 g) in THF (8.6 kg), maintaining the temperature at −15° to −5° C. Water was added and following removal of the THF solvent by distillation the product 3,5-bis(trifluoromethyl)acetophenone was isolated by steam distillation and fractionally distilled to yield 1 kg (99% pure).

### EXAMPLE 3

3,5-Bis(trifluoromethyl)bromobenzene, (196 g), in THF, (400 mL), was fed to a slurry of magnesium turnings, (17.4 g), in THF, (100 mL). The temperature was maintained at approximately 45° C. The solution of Grignard reagent was fed to a mixture of acetyl chloride, (59 g), and cuprous chloride, (4 g), in THF, (150 mL), maintaining the temperature at 30-40° C. Water was added and, following the removal of the THF solvent by distillation, the product 3,5-bis(trifluoromethyl)acetophenone was isolated by steam distillation and fractionally distilled to yield 100.3 g (99% pure).

### EXAMPLE 4

3,5-Bis(trifluoromethyl)bromobenzene, (196 g), in THF, (400 mL), was fed to a slurry of magnesium turnings, (17.4 g), in THF, (100 mL). The temperature was maintained at approximately 45° C. The solution of Grignard reagent was fed to a mixture of acetyl chloride, (59 g), and cuprous chloride, (67.6 g), in THF, (150 mL), maintaining the temperature at less than 30° C. Water was added and, following the removal of the THF solvent by distillation, the product 3,5-bis(trifluoromethyl)acetophenone was isolated by steam distillation and fractionally distilled to yield 106.7 g (99% pure).

### EXAMPLE 5

3,5-Bis(trifluoromethyl)acetophenone, (500 g) containing approximately 0.5% of 3,5-bis(trifluoromethyl)phenyl acetate, was heated at reflux for 1.5 hours with 1N sodium hydroxide solution, (100 mL). The mixture was cooled, the aqueous phase was separated off and the organic phase was washed free of alkali with water. Levels of the impurity were reduced to less than 0.05%. The product was then fractionally distilled.

It is to be understood that the above described embodiments of the invention are by way of illustration only. Many modifications and variations are possible.

1. A method of manufacture of 3,5-bis(trifluoromethyl)bromobenzene, comprising the addition of a brominating reagent to a mixture of 1,3-bis(trifluoromethyl)benzene together with at least one of sulphuric acid or oleum in the absence of acetic acid.

2. A method according to claim 1, wherein the brominating reagent is selected from elemental bromine, N-bromosuccinimide and 1,3-dibromo-5,5 dimethylhydantoin.

3. A method according to claim 1 or claim 2, wherein one equivalent of brominating reagent is used.

4. A method according to any of claims 1 to 3, wherein the brominating reagent is added portion-wise.

5. A method according to any preceding claim, wherein at least 3 parts by weight of the acid and/or oleum is used to one part of the 1,3-bis(trifluoromethyl)benzene.

6. A method according to claim 5, wherein 4 parts by weight of the acid and/or oleum is used to one part of the 1,3-bis(trifluoromethyl)benzene.

7. A method according to any of claims 1 to 6, wherein the acid is not diluted to less than 90% solution.

8. A method according to claim 7, wherein the acid is 96 to 98% solution.

9. A method according to any preceding claim, wherein the reaction is carried out at a temperature in the range from −10° C. to 30° C.

10. A method according to claim 9, wherein the reaction is carried out at a temperature in the range from 0° C. to 10° C.

11. A method according to claim 10, wherein the reaction is carried out at a temperature in the range from 3° C. to 5° C.

12. A method according to any of claims 1 to 8, wherein the reaction temperature is initially within the range from 3° C. to 5° C. and is then allowed to rise to ambient temperature.

13. A method according to any preceding claim further comprising at least one subsequent step of washing.

14. A method according to claim 13, wherein at least one subsequent step of washing is carried out using bisulphite.

15. A method according to any preceding claim further comprising at least one purification step.

16. A method according to claim 15, wherein the at least one purification step is a fractional distillation.

17. A method of production of 3,5-bis(trifluoromethyl)acetophenone comprising the reaction of 3,5-bis(trifluoromethyl)phenyl magnesium bromide with acetyl chloride in the presence of cuprous chloride.

18. A method according to claim 17, wherein the cuprous chloride in present in a catalytic amount.

19. A method according to claim 17, wherein the cuprous chloride is present in an equimolar amount to the acetyl chloride.

20. A method according to claim 17, wherein the reaction temperature is within the range from 30° C. to 40° C.

21. A method according to claim 17, wherein the reaction temperature is maintained below 30° C.

22. A method according to any of claims 17 to 21, wherein the acetyl chloride is in an organic solvent.

23. A method according to claim 22, wherein the organic solvent is tetrahydrofuran.

24. A method according to any of claims 17 to 23, wherein the 3,5-bis(trifluoromethyl)acetophenone is isolated by the addition of water followed by steam distillation.

25. A method according to claim 24, comprising the further step of fractional distillation.

26. A method of production of 3,5-bis(trifluoromethyl)acetophenone comprising the steps of reacting 3,5-bis(trifluoromethyl)phenylmagnesium bromide with acetic anhydride, adding water and recovering the product by azeotropic distillation.

27. A method according to claim 26, wherein the acetic anhydride is in an organic solvent.

28. A method according to claim 27, wherein the organic solvent is tetrahydrofuran.

29. A method according to any of claims 26 to 28, wherein an excess of acetic anhydride is used.

30. A method according to claim 29, wherein less than 1.5 equivalents of acetic anhydride are used.

31. A method according to claim 30, wherein less than 1.1 equivalent of acetic anhydride are used.

32. A method according to any of claims 26 to 31, wherein the reaction temperature is within the range from −15° C. to +15° C.

33. A method according to claim 32, wherein the reaction temperature is within the range from −10° C. to −5° C.

34. A method according to any of claims 26 to 33 comprising the further step of fractional distillation.

35. A method of removal of impurities including 3,5-bis(trifluoromethyl)phenyl acetate, 4-bromobutyl acetate and 4-chlorobutyl acetate from a preparation of 3,5-bis(trifluoromethyl)acetophenone, the method comprising heating the 3,5-bis(trifluoromethyl)acetophenone with a dilute solution of alkali.

36. A method according to claim 35, wherein the 3,5-bis(trifluoromethyl)acetophenone is heated at reflux with the dilute solution of alkali.

37. A method according to claim 35 or claim 36, wherein the alkali is sodium hydroxide.

38. A method according to claim 37, wherein the sodium hydroxide comprises a 1N solution.

39. A method according to any of claims 35 to 38, wherein the 3,5-bis(trifluoromethyl)acetophenone is heated with the dilute solution of alkali for at least 30 minutes.

40. A method of production of 3,5-bis(trifluoromethyl)phenyl magnesium bromide comprising the reaction of 3,5-bis(trifluoromethyl)bromobenzene with magnesium in a solvent whilst maintaining the temperature of the reactants above 20° C. and below the reflux temperature of the solvent.

41. A method according to claim 40, wherein the solvent is selected from diethyl ether, dimethoxyethane, butyldiglyme, 2-methyl tetrahydrofuron and tetrahydrofuron.

42. A method according to claim 40 or claim 41, wherein the temperature is maintained within the range from 30° C. to 60° C.

43. A method according to claim 42, wherein the temperature is maintained within the range from 35° C. to 50° C.

44. A method according to claim 43, wherein the temperature is maintained at approximately 45° C.
